(19) United States
(12) Patent Application Publication
(10) Pub. No.: US 2026/0096961 A1
(43) Pub. Date: Apr. 9, 2026

(54) ANTIPERSPIRANT COMPOSITIONS

(71) Applicant: The Procter & Gamble Company, Cincinnati, OH (US)

(72) Inventors: David Frederick Swaile, Cincinnati, OH (US); Wade Charles Henke, Mason, OH (US); Amanda Swee Chan Chew, Chicago, IL (US); Stephen John Provancal, Chicago, IL (US); Elton Luis Menon, Mason, OH (US); Richard Oryszczak, Palatine, IL (US)

(21) Appl. No.: 19/349,063

(22) Filed: Oct. 3, 2025

Related U.S. Application Data
(60) Provisional application No. 63/702,962, filed on Oct. 3, 2024.
(60) Provisional application No. 63/875,727, filed on Sep. 4, 2025.

(51) Int. Cl. A61K 8/26; A61K 8/20; A61K 8/44; A61Q 15/00

(52) U.S. Cl. CPC A61K 8/26; A61K 8/20; A61K 8/44; A61Q 15/00

(57) ABSTRACT

A particulate active premix composition that includes basic aluminum chloride of formula Al₂OH_{5.05}Cl_{0.95} to Al₂OH_{4.95}Cl_{1.05}, calcium chloride, glycine, and greater than 1% of an alkali metal salt and/or magnesium chloride. The composition can be a particle. The particulate active premix can be incorporated into antiperspirant products.

### FIELD OF THE INVENTION

The present disclosure relates to an antiperspirant composition and methods relating thereto. More particularly, the antiperspirant composition is a plurality of particles that includes a basic aluminum chloride of formula Al₂(OH)_{5.05}Cl_{0.95} to Al₂(OH)_{4.95}Cl_{1.05}, calcium chloride, glycine, and greater than 1% of an alkali metal salt.

### BACKGROUND OF THE INVENTION

An antiperspirant is a personal care product designed to reduce or prevent sweating to help control body malodor. Its primary function is to inhibit or block the sweat glands, thereby reducing the amount of perspiration produced by the body. Along with controlling sweat, antiperspirants often contain deodorizing agents that help mask or neutralize the malodor associated with perspiration. Antiperspirants are typically applied topically to the underarms or other areas of the body prone to sweating.

Aluminum-zirconium (Al—Zr) compounds, such as aluminum zirconium tetrachlorohydrex gly, are common antiperspirant actives because they effectively reduce sweat production. However, it can be desirable to use aluminum-only active ingredients because zirconium can be expensive, subject to regulatory restrictions, and cannot be used in aerosol spray formulations because there are safety concerns associated with inhaling aerosolized zirconium compounds.

There are antiperspirant actives that are aluminum-based, such as the basic aluminum chlorides, aluminum chlorohydrate, aluminum sesquichlorohydrate, and aluminum dichlorohydrate. However, they tend to have reduced efficacy as compared to Al—Zr compounds.

The effectiveness of aluminum-only actives can be enhanced through various treatments, such as thermal activation and/or incorporation of amino acids like glycine (gly) and alkaline earth metal salts, like calcium chloride and strontium chloride. These treatments aim to stabilize the active salt in solution. However, these treatments only provide limited improvement in effectiveness and are primarily used commercially with more acidic aluminum-based actives, such as aluminum sesquichlorohydrate.

Aluminum sesquichlorohydrate is generally more effective when compared to activated aluminum chlorohydrate particulate actives. However, its low pH can have drawbacks. For instance, the low pH can degrade fragrances, which are crucial for providing freshness and masking malodors. Additionally, the acidic aluminum sesquichlorohydrate can cause corrosive damage to metal processing equipment and if these actives are utilized in aerosol products, the metal components of the aerosol dispenser can be susceptible to corrosive damage.

Therefore, there is a need for an aluminum-based antiperspirant active that not only demonstrates high-efficacy but also maintains fragrance stability and, in the case of aerosol products, ensures stability of the aerosol container.

### SUMMARY OF THE INVENTION

A composition comprising: (a) basic aluminum chloride of formula Al₂(OH)_{5.05}Cl_{0.95} to Al₂(OH)_{4.95}Cl_{1.05}; (b) calcium chloride; (c) glycine; (d) greater than 1% of an alkali metal salt; wherein the composition is a particle.

A method of making a particulate antiperspirant active premix comprising: (a) providing an aqueous solution of aluminum chlorohydrate with the formula Al₂(OH)_{5.05}Cl_{0.95} to Al₂(OH)_{4.95}Cl_{1.05}; (b) adding calcium chloride to provide a calcium to aluminum molar ratio of at least 0.05 and adding glycine to provide a glycine to calcium molar ratio from about 1 to 2 to form an aqueous solution; (c) adding sodium chloride to the aqueous solution at a concentration of more than 0.5% to form the aqueous premix solution; (d) heating the aqueous premix solution at a temperature of more than 60° C. for more than 1 hour; (e) after step (d), drying the aqueous premix solution to a plurality of particles, forming the particulate antiperspirant active premix; and (f) optionally grinding the particulate antiperspirant active premix to a Dv50 particle size less than 50 microns.

A method of making a particulate antiperspirant active premix comprising: (a) providing an aqueous solution of aluminum chlorohydrate Al₂(OH)_{5.05}Cl_{0.95} to Al₂(OH)_{4.95}Cl_{1.05}; (b) adding calcium chloride to provide a calcium to aluminum atomic ratio of at least 0.05 and adding glycine to provide a glycine to calcium molar ratio from about 1 to 2 to form an aqueous solution; (c) heating the aqueous solution at a temperature of more than 60° C. for more than 1 hour; (d) after step (c), adding sodium chloride at a concentration of more than 0.5% of the aqueous solution; (e) drying the aqueous solution to a particle, forming the particulate antiperspirant active premix; and (f) optionally grinding the particulate antiperspirant active premix to a Dv50 particle size less than 50 microns.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1A shows an image from a scanning electron microscope of a particle from an active premix without a surface coating;

FIG. 1B shows an image from a scanning electron microscope (SEM) of a particle from an active premix with a surface coating with sodium chloride;

FIG. 2 shows the measured change in mass vs. time for dynamic vapor sorption measurements of Example 1 and Comparative Example 3; and

FIG. 3 is a representative chromatogram for Peaks I-V.

### DETAILED DESCRIPTION OF THE INVENTION

Aluminum-zirconium (Al—Zr) compounds, such as aluminum zirconium tetrachlorohydrex gly, are common, efficacious antiperspirant actives. However, aluminum-only active ingredients can be preferred over aluminum-zirconium compounds due to cost, regulatory restrictions, and safety concerns associated with zirconium inhalation in aerosol spray formulations. There are antiperspirant actives that contain aluminum-only. However, they tend to have reduced efficacy as compared to Al—Zr compounds.

The effectiveness of aluminum-only actives can be enhanced through various treatments. One treatment is heating before spray drying to increase the concentration of smaller, more efficacious polymers in the active. In aluminum-only and Al—Zr complexes, these smaller polymers are often identified and referred to as band 3 or as peak 4 (U.S. Pat. Nos. 4,359,456 and 10,526,210 describe heating aluminum active solutions). The relative concentration of these polymers is typically quantified as either the band III/II ratio or Peak 4/3 ratio). Ratios exceeding 0.75 are generally indicative of a more effective active ingredient. This enhancement process is commonly termed “activation,” and the resulting formulations are described as “activated,” “enhanced,” or “improved” AP actives.

Many companies have tried to develop and market effective aluminum-only antiperspirant actives. One example is an aluminum chlorohydrate solution that is activated and stabilized by the addition of calcium chloride and an amino acid that can be incorporated into a wax stabilized water-in-oil emulsion, which was previously marketed in Old Spice® Ever Clear an aqueous antiperspirant stick manufactured by Procter & Gamble® containing ˜30-40% water. This active solution had a calcium to aluminum atomic ratio of 0.06 and a glycine to calcium molar ratio of 5.2. These ratios were intended to provide long term solution stability of the activated active. The Old Spice® Ever Clear product was not as well-received by consumers because they generally prefer the dry feel of anhydrous products and this active was only stable in aqueous-based products.

Attempts to generate a highly effective particulate active premix by spray drying an aluminum chlorohydrate, calcium chloride and glycine solution were unsuccessful and were not marketed by Procter & Gamble®. It is believed that spray drying the aluminum chlorohydrate, calcium chloride, and glycine solution did not provide a hygroscopic enough particulate active premix to dissolve rapidly in sweat, thus lowering overall antiperspirant efficacy.

Another example is aluminum sesquichlorohydrate active premixes that are activated by calcium chloride and glycine before spray drying by Unilever®, which is used in Degree® Advanced Antiperspirant Dry Spray Deodorant products (see U.S. Pat. Nos. 10,632,052; 10,660,830; 10,682,293; and 10,729,627).

Aluminum sesquichlorohydrate actives can be preferred over aluminum chlorohydrate actives because they tend to be more hygroscopic, which enables a rapid dissolution rate and incorporation into anhydrous products. However, aluminum sesquichlorohydrate actives are more acidic than aluminum chlorohydrate actives. As a result, they can cause corrosive damage to metal processing equipment and metal components of the aerosol dispenser (if present); notably the internal surfaces of the can, the valve cup, and metal spring in the valve can degrade fragrance components.

It was found that a particulate active premix containing aluminum chlorohydrate of formula Al₂(OH)_{5.05}Cl_{0.95} to Al₂(OH)_{4.95}Cl_{1.05}, (Al:Cl atomic ratios between about 1.91 and about 2.1) and amino acids, alkaline earth metal salts and an alkali metal salt can have enhanced hygroscopicity and antiperspirant efficacy. Additionally, the active premix can have higher pH values compared to other hygroscopic actives, which can improve compatibility with a wide array of perfumes, additives, anhydrous formulas, processing equipment, and containers.

To generate the active premix, combine aluminum chlorohydrate, amino acids, alkaline earth metal salts, and alkali metal salts in an aqueous solution. The solution is then heated and subsequently dried to a powder premix. Alternatively, the alkaline earth metal salts can be added after heating but before drying to form a powder premix.

Each particle in the active premix can include aluminum, chlorine, and sodium. The sodium can be interspersed with the aluminum and chloride and/or the sodium can be present in a surface coating. This can be visualized using energy-dispersive X-ray (EDX) spectroscopy coupled with a scanning electron microscope (SEM).

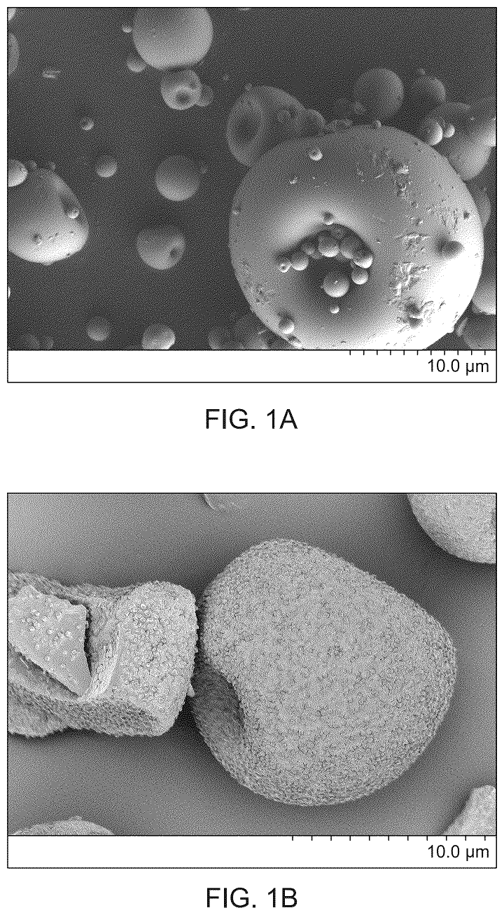

Both FIGS. 1A and 1B show images from an SEM microscope of particles from an active premix containing aluminum chlorohydrate, represented by the formula Al₂(OH)_{5.05}Cl_{0.95} to Al₂(OH)_{4.95}Cl_{1.05}, along with glycine, and calcium. In FIG. 1A, the particles lack a sodium chloride surface coating, resulting in a smooth appearance. In contrast, FIG. 1B shows particles from an active premix that does include sodium chloride. These particles exhibit a rough surface texture, with visible cubic sodium chloride crystals, indicating the presence of the sodium chloride coating.

It was surprising to find sodium chloride deposits on the particle surfaces, especially since the spray-dried mixture is homogeneous and contains relatively low sodium compared to the amounts of aluminum. In some instances, the sodium chloride covers a significant portion of the particle surface (e.g., more than a majority, >80% up to 95%) while in other cases, only about 50% may be covered.

The active premixes can have an amino acid to alkaline earth metal molar ratio of <5, <4, <3, ≤2, ≤1.75, ≤1.5, or ≤1.4. The active premixes can have an amino acid to alkaline earth metal molar ratio of ≥0.25, ≥0.5, ≥0.75, ≥0.90, ≥1.0, ≥1.1, ≥1.25, or ≥1.3. Without wishing to be bound by theory, it is believed that lower ratios can be more effective in producing active premixes with improved hygroscopic properties. On the other hand, higher ratios are believed to be beneficial in achieving longer periods of solution stability before drying the active premix to a particle and it can be desirable to adjust the ratio based on manufacturing conditions.

The active premix can have a pH of about 4.2 to about 5.3, from about 4.3 to about 5.2, from about 4.4 to about 5.1, from about 4.5 to about 5.0, from about 4.6 to about 4.9, from about 4.7 to about 4.8, or about 4.7, as determined by the pH Test Method, described herein. The active premix can have a pH of about 4.2 to about 5, 4.3 to about 4.9, or from about 4.4 to about 4.8.

The active premix can have a maximum change in mass over 48 hours of ≥40%, ≥42%, ≥45%, ≥48%, ≥50%, ≥52%, ≥54%, ≥60%, ≥70%, or ≥80% according to the Water Vapor Sorption Method, described herein. In order to maintain the stability and processability required for practical application in manufacturing antiperspirant formulations and shipping of antiperspirant formulations and active salts it can be advantageous for the active premix to have a change in mass over 48 hours of ≤150%, ≤140%, ≤130%, ≤125%, ≤115%, ≤110%, according to the Water Vapor Sorption Test Method, described herein.

Aluminum chlorohydrate (ACH) can include a complex basic aluminum chloride that is polymeric and loosely hydrated. This compound includes a range of aluminum to chloride atomic ratios from about 1.91:1 to about 2.10:1. Aluminum chlorohydrate can also be represented by the empirical formula range of Al₂(OH)_{5.05}Cl_{0.95} to Al₂(OH)_{4.95}Cl_{1.05}. Other characteristics of aluminum chlorohydrate can be found in the United States Pharmacopoeia and National Formulary (USP/NF) in its monograph (2024).

The polymer size distribution of the ACH in the active premix can be defined by size exclusion chromatography method using Gel Permeation Chromatography (GPC), described herein in the GPC Test Method. The active premix can have a peak IV/III (Band III/II) ratio of exceeding 0.75, ≥1, ≥1.5, ≥2, ≥2.1, ≥2.4, ≥2.5, ≥3, ≥3.25, ≥3.4, ≥3.5, ≥3.75, ≥3.85, ≥3.90, ≥4.0, ≥4.2, ≥4.4, ≥4.6, ≥4.8, ≥5.0, ≥5.2, ≥5.4, ≥5.6, ≥5.8, ≥6.0, ≥6.25, ≥6.5, ≥6.75, ≥7.0, ≥7.25, ≥7.5, ≥7.75, or ≥7.9 according to the GPC Test Method. Active premixes having a peak IV/III ratio exceeding 0.75 can be referred to as “activated” and the process of heating to increase the peak IV/III ratio can be referred to as “activation.” The peak IV/III ratio can be dependent on several factors including the level and type of alkaline earth metal salt, amount and type of amino acid, and temperature and time of heating in solution prior to drying.

Alkaline earth metal salts can include an alkaline earth metal cation and an anion that is acceptable for application to the skin. Alkaline earth metals are elements in Group 2 of the periodic table and include beryllium, magnesium, calcium, and strontium. The anions associated with the alkaline earth metals salts can include chloride, sulfate, nitrate, or other halogen anions. More specifically, the salts can include calcium chloride, calcium nitrate, calcium sulfate, strontium chloride, strontium sulfate and strontium nitrate. Among these, calcium chloride may be preferred. The composition can include from about 0.5% to about 13%, from about 1% to about 10%, from about 1.5% to about 7%, or from about 1.75% to about 5% alkaline earth metal salts.

The amino acids can include natural amino acids, unnatural amino acids (e.g., non-canonical), and/or derivatives thereof (e.g., betaines such as trimethylglycine). The amino acids can include alanine, arginine, asparagine, aspartic acid, beta-alanine, cysteine, glutamic acid, glutamine, glycine, trimethylglycine, histidine, isoleucine, leucine, lysine, methionine, phenylalanine, proline, serine, threonine, tryptophan, tyrosine, valine, or mixtures thereof. Among these, glycine, trimethylglycine, alanine, leucine, or mixtures thereof may be preferred. Among these, glycine, alanine, and/or trimethylglycine may be preferred. The composition can include from about 5% to about 25%, from about 6% to about 17%, from about 7% to about 14%, from about 9% to about 13%, or from about 9.4% to about 11.5% of the amino acid.

It was found that active premixes containing glycine, alanine, beta-alanine, or trimethylglycine exhibited similar performance characteristics when tested in vitro. It is believed that these amino acids, along with others, would be acceptable to consumers in the active premixes described herein. Additionally, some consumers may prefer naturally derived amino acids, including trimethylglycine.

Moreover, the active premixes can have an alkaline earth metal to aluminum atomic ratio ≥0.05, ≥0.07, ≥0.09, or ≥0.10. The active premixes can have an alkaline earth metal to aluminum atomic ratio of ≤1, ≤0.75, ≤0.5, ≤0.3, ≤0.2, or 0.17. The active premixes can have an alkaline earth metal to aluminum atomic ratio of about 0.05 to about 0.5, about 0.07 to about 0.3, or about 0.1 to about 0.2. Without wishing to be bound by theory, it is believed that higher ratios can create more activation or allow activation at lower temperatures. This higher ratio may also provide longer solution stability of the polymers before the active premix is dried to a particle. However, the higher ratio can generate higher absolute levels of glycine in the active premix that may, in turn, lead to a less hygroscopic active premix.

The active premixes can also include an alkali metal salt that is acceptable for use on skin. Alkali metals are elements in Group 1 of the periodic table and can include: lithium, sodium, potassium, or mixtures thereof. Anions for these salts of these metals can include chloride, sulfate, phosphates, nitrate, other halogen anions, or mixtures thereof. Acceptable salts can include sodium chloride, sodium sulfate, sodium phosphate, sodium nitrate, lithium chloride, lithium sulfate, lithium phosphate, lithium nitrate, potassium chloride, potassium nitrate, potassium sulfate, potassium phosphate, or mixtures thereof. The salts can include sodium chloride, potassium chloride, lithium chloride, or mixtures thereof. Among these, sodium chloride may be preferred. Without wishing to be bound by theory, it is believed that salts can further increase the water dissolution rate of the active premix. These salts can comprise up to 25% of the active premix, less than 15%, or less than 10%. If present, these salts can comprise at least 1% of the active premix, at least 2%, at least 3%, at least 4%, or at least 5%.

The active premixes can be made according to the following method: A solution that includes ACH, the amino acid, and the alkaline earth metal can be heated to be activated, which is indicated by having a peak IV/III ratio of exceeding 0.75. The time and temperature of heating will be dependent on the concentration of ACH in solutions during heating, the alkaline earth metal mole ratio, and the amino acid to alkaline earth metal molar ratio. Heating temperatures are 70° C. to 95° C. and heating time range from 1-24 hours. Heating temperatures, heating times, the alkaline earth metal atomic ratio, and the amino acid to alkaline earth metal molar ratio can be varied based on desired peak IV/III ratio, desired degree of hygroscopicity and available processing equipment. If present, the alkali metal salt can be added before or after heating.

The particulate active premixes can be dried by any suitable manner including belt drying, tray drying and spray drying. Among these, spray drying may be preferred. The dried particle may be ground to desired particle size and/or sieved to desired particle distribution and may be spherical, platelet or random mixture in shape. The particle size may vary based on the final product (e.g., spray, cream, roll-on or stick product). The particulate active premix could be generally spherical in shape and can have a Dv50 average particle size of 25-35 microns. The particulate active premix could be generally platelet in shape and have a Dv50 average particle size of 12 to 18 micron. The active could be finely ground to have more than 85% or more than 90% (Dv90) of the particle having a size less than 10 microns and an average of less than 6 microns. The active premix powder can also be dissolved or suspended in a solution, including aqueous, propylene glycol, propylene carbonate, or alcohol-based solution.

### EXAMPLES

Comparative Example 3 in Table 1, below, was made as follows: aluminum chlorohydrate solution (% Al: 12.79, % Cl: 8.30 concentration), calcium chloride solution (31.39% concentration), and glycine were added to a 5 L reactor equipped with a mechanical stirrer, reflux condenser, and temperature controller. The reactor was then brought to 95° C. and the solution was stirred for at least one and up to 24 hours. The reactor was then cooled to room temperature, and the solution was filtered through a 0.7 μm filter. The filtrate solution was collected, and spray dried to isolate a powder. Spray drying was performed with a Bowen Engineering, INC. (BE-994) Spray Dryer using the following conditions: Inlet temperature: 205° C., Outlet Temperature 130-140° C., air pressure: 60 PSI, and Collector H₂O: 6.5 in. The collected powder was then milled using a Hosokawa Alpine® jet mill (model #: 30369) with the following particle size distribution: D₅₀: 14.1 microns, D₉₀: 24.5 microns, and 0% greater than 88 microns to form the active premix.

Example 1 in Table 1, was made similarly to Comparative Example 3 except: (1) sodium chloride was added to the reactor prior to heating and (2) the powder that was collected after spray drying was milled to the following particle size distribution: D₅₀: 13.4 microns, D₉₀: 23.3 microns, and 0% greater than 88 microns to form the active premix.

Example 2 in Table 1, was made similarly to Comparative Example 3 except: (1) after the reactor is cooled to ambient temperature and before the solution is filtered, 0.4 kg water and 0.1 kg sodium chloride were added to the reactor and stirred for 30 min at ambient temperature and (2) the powder that was collected after spray drying was milled to the following particle size distribution: D₅₀: 14.0 microns, D₉₀: 24.4 microns, and 0% greater than 88 microns to form the active premix.

TABLE 1

| | | | Comp. | Comp. | Comp. |
|---|---|---|---|---|---|
| | Ex. 1 | Ex. 2 | Ex. 1 | Ex. 2 | Ex. 3 |
| | Active | Active | Active | Active | Active |
| | Premix | Premix | Premix | Premix | Premix |
| | (wt. %) | (wt. %) | (wt. %) | (wt. %) | (wt. %) |
| Aluminum | 20.39 | 20.66 | 24.22 | 17.2 | 21.27 |
| Chloride | 23.91 | 23.83 | 20.37 | 23.8 | 21.65 |
| USP Anhydrous | 65.9 | 66.5 | 80.63 | 59.38 | 68.8 |
| Active Level | | | | | |
| (USP Assay) | | | | | |
| Ca | 4.07 | 4.18 | | 2.97 | 4.25 |
| Glycine | 10.1 | 10.4 | | 16.65 | 10.8 |
| Al:Cl atomic ratio | 2.02 | 2.09 | 1.5 | 1.22 | 2.00 |
| % Na | 3.24 | 3.26 | | | |
| % NaCl | 5.37 | 5.40 | | | |
| Ca:Al atomic ratio | 0.13 | 0.14 | | | 0.13 |
| Gly:Ca molar ratio | 1.32 | 1.33 | | | 1.36 |
| Active pH (15% | 4.7 | 4.7 | 3.3 | 3.4 | 4.8 |
| solution in water) | | | | | |
| Peak 4/3 ratio | 2.9 | 3.2 | 1.07 | | 3.3 |

### Kinetic Study

A kinetic study was performed on Examples 1 and Comparative Example 3 in Table 1 according to the Water Vapor Sorption Test Method, described hereafter. The test determined the amount of water vapor sorption that occurs in a raw material.

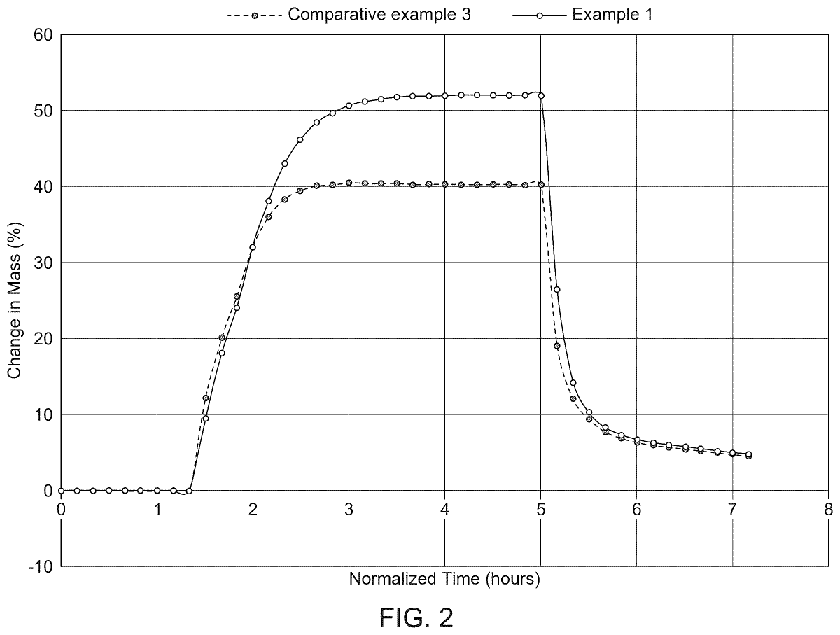

FIG. 2 shows that Example 1 had a significantly higher moisture increase over time as compared to Comparative Example 3. This indicates that sodium chloride added to Example 1 increases its hygroscopicity versus Comparative Example 3, which is the same active, without sodium chloride. This effect is expected to increase the rate of dissolution in sweat thereby improving antiperspirant efficacy.

### Clinical Study

A randomized, double-blind, 4-treatment round-robin study with 36 female subjects was performed determine whether Comparative Examples A-B and D and Example C, described in Table 2 and Table 3, below, had in vivo efficacy. Subjects underwent a 17-day washout with marketed bodywash, Ivory® Unscented, for all bathing and use of Old Spice® Fiji body spray as deodorant. Subjects were instructed to not use any antiperspirant products in their underarms during washout, and this was confirmed by swabbing the axilla for presence of aluminum at the baseline visit. The female subjects were required to shave underarms 3 days prior to the baseline visit.

Hot room sessions (40° C., 35% RH) were conducted at baseline and 72-hours after the fourth treatment. Hot room sessions included a 40-minute seated sweat-collection, followed by two, 20-minute walking sessions with sweat collection. Subjects who produced ≥250 mg and ≤1100 mg of sweat/axilla for the average of both 20-minute sweat collections, and who had a right/left axillary sweat ratio in the range of 0.6 to 1.67 at the baseline visit were randomized to treatment. A 4-day treatment phase followed, where products were applied once a day, on-site using a two second application dose, in the morning, followed by a 72-hour hot room visit after the fourth application.

The average of the two 20-minute sweat collections were analyzed using a mixed model Ancova (Baseline, Side and Treatment as fixed and Subject as random) after a base 10 logarithm transformation of the data. Descriptive statistics (N, least-squares mean, standard error and 95% two-sided confidence intervals) were calculated by treatment group and evaluated by two-sided hypothesis testing with type I error rate of 5%. The product concentrate pH was determined according to the pH method, described herein.

TABLE 2

| | Compar- | Compar- | |
|---|---|---|---|
| | ative | ative | |
| | Product A | Product B | Example C |
| Raw Material Name | (wt %) | (wt. %) | (wt. %) |
| Propellant Blend: | 60.00 | 60.00 | 60.00 |
| 61.67% A-17 propellant (n- | | | |
| Butane@>95%/ | | | |
| Isobutane@<5%) | | | |
| 38.33% hydrofluorocarbon 152A | | | |
| Cyclopentasiloxane | 21.95 | 21.95 | 21.95 |
| Comparative Example 1 | 11.40 | | |
| Comparative Example 2 | | 11.40 | |
| Example 1 | | | 11.40 |
| PPG-14 Butyl Ether | 3.80 | 3.80 | 3.80 |
| Dimethicone (and) Dimethiconol | 0.38 | 0.38 | 0.38 |
| Disteardimonium Hectorite | 1.14 | 1.14 | 1.14 |
| Mineral Oil Light White | 0.19 | 0.19 | 0.19 |
| Propylene Carbonate | 0.38 | 0.38 | 0.38 |
| Envicap Aerosol Starch AN with | 0.76 | 0.76 | 0.76 |
| Feminine 4 2017C | | | |
| Anhydrous active in product | 23.1 | 18.5 | 18.5 |
| concentrate | | | |
| Product Concentrate pH | 4 | 4.0 | 4.8 |

TABLE 3

| Comparative Product D | INCI |
|---|---|
| Degree ® Advanced Antiperspirant | Active Ingredient: Aluminum Sesquichlorohydrate |
| Dry Spray Deodorant Dry Spray | (17.6%) |
| Sexy Intrigue ® | Inactive Ingredients: Butane, Cyclopentasiloxane, |
| Lot code 01034AX02 | Hydrofluorocarbon 152a, Isobutane, PPG-14 Butyl |
| Exp. January 2026 | Ether, Glycine, Fragrance (Parfum), Disteardimonium |
| | Hectorite, Calcium Chloride, Propane, BAHT, |
| | Propylene Carbonate, Sodium Starch |
| | Octenylsuccinate, Maltodextrin, Hydrolyzed Corn |
| | Starch Octenylsuccinate, Silica. |
| Product Concentration pH | 4.5 |

The results from the study are in Table 4, below. It was found that Ex. A (containing aluminum sesquichlorohydrate) and Ex. B (containing aluminum dichlorohydrate) did not perform as well as Ex. C (containing the inventive active premix) or Lx. D (Degree® product containing aluminum sesquichlorohydrate).

TABLE 4

| | | Reduction | vs. Ex. B | vs. Ex. C | vs. Ex. C |
|---|---|---|---|---|---|
| | Sweat | from | Two-sided | Two-sided | Two-sided |
| | (mg) | Baseline | P-value | P-value | P-value |
| Comp. Ex. A | 293 | −33.6% | 0.7760 | 0.0049 | 0.0299 |
| Comp. Ex. B | 299 | −32.1% | | 0.0029 | 0.0168 |
| Ex. C | 232 | −47.4% | | | 0.4585 |
| Comp. Ex. D | 246 | −44.3% | | | |

Ex. C was statistically better at reducing sweat, as compared to Comp. Lx. A and B. Lx. C and Comp. Ex. D were shown to have similar performance. However, Comp. Ex. D has a lower pH than Lx. C, which may be required for the stability of the aluminum sesquichlorohydrate active. However, this low pH can degrade fragrance and aerosol containers and therefore Lx. C may be preferred over Comp. Ex. D.

Premix Examples X and Y in Table 5 were made according to the methods described herein.

TABLE 5
Premix Examples

| | Ex. X | Ex. Y |
|---|---|---|
| % Al | 8.95-9.16 | 18.15-19.97 |
| % Ca | 1.68-2.37 | 3.62-5.01 |
| % Glycine | 3.94-5.55 | 7.95-11.59 |
| % Na | 0.75-1.06 | 1.67-2.32 |
| % Cl | 10.29-11.83 | 21.98-24.83 |
| Active pH | 4.43-4.79 | 4.37-4.79 |
| GPC Peak 4:3 Ratio | 2.49-3.92 | 2.17-3.44 |
| Gly:Ca Molar Ratio | 0.89-1.66 | 0.85-1.68 |
| Ca:Al Atomic Ratio | 0.13-0.18 | 0.12-0.18 |

### Methods of Use

The active premix particles can be incorporated into antiperspirant products. The product can be a spray, a roll-on, cream, paste, foam, wipes, or a stick including anhydrous sticks, gel sticks including aqueous gel sticks, soft solids, or glycol-based sticks. The antiperspirant product may be topically applied to the axilla or other area of the skin in any known or otherwise effective method for controlling malodor associated with perspiration. These methods comprise applying to the axilla or other area of the human skin an effective amount of an antiperspirant product that includes the active premix composition, typically about 3.5 to about 5 mg/cm² of the product, and more typically about 4 mg/cm². The product is generally a leave-on composition that can provide lasting odor protection and freshness and the composition may provide 72 hours or more of odor control after application. The product can provide wetness protection, which can refer to the product's ability to help reduce or prevent excessive sweating and the resulting wetness under the arms.

### Definitions

Except as otherwise noted, the articles “a”, “an”, and “the” mean “one or more”.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

All numbers expressing pH values are to be understood as being modified by the term “about,” and as encompassing readings using a pH meter having a variation of up to +10%, such as up to +9%, up to +8%, up to +7%, up to +6%, up to +5%, up to +4%, up to +3%, up to +2%, or up to +1%, which a skilled person will recognize relates to the inherent variation in pH meters.

As used herein, the expressions “ranging from” and “between” are inclusive of the endpoints of the recited range(s).

All percentages are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Test Methods

### GPC Test Method

The GPC Test Method is performed on sample active premix to quantify relative concentration of aluminum polymer populations present. The method is preferably performed on active or active premix but can also be performed on finished antiperspirant product if active or active premix is not available. Peaks corresponding to five characteristic polymer populations are identified and integrated, and ratios of relative areas of peaks of interest are calculated and reported.

### Sample Preparation

If sample active or active premix is available, it is dissolved or dispersed in 0.01-M nitric acid such that active is present at 1.0% w/w. If only sample finished antiperspirant product is available, it is dissolved or dispersed in 0.01-M nitric acid such that the active is present at 1.0% w/w. After dilution, the nitric acid solution may be filtered (for example, using a 0.45-μm nylon syringe filter) if necessary. In any case, only the resulting nitric-acid extraction solution is subsequently injected into the chromatograph.

### Apparatus and Procedure

GPC chromatographic separation is performed using 0.01-M nitric acid mobile phase, using a total 900 mm length of silica columns. Exemplary suitable apparatus consists of three consecutive μPorasil Columns, 3.9×300 mm, 10 mm packing (available from Waters, Milford, Massachusetts), plumbed in series. The chromatograph is equipped with a refractive-index detector. Exemplary suitable refractive-index detector is ERC RefractoMax 520 (available from IDEX, Oak Harbor, Washington). A 5-μL injection volume is used, and an exemplary suitable flow rate is 0.8 mL/min.

### Analysis

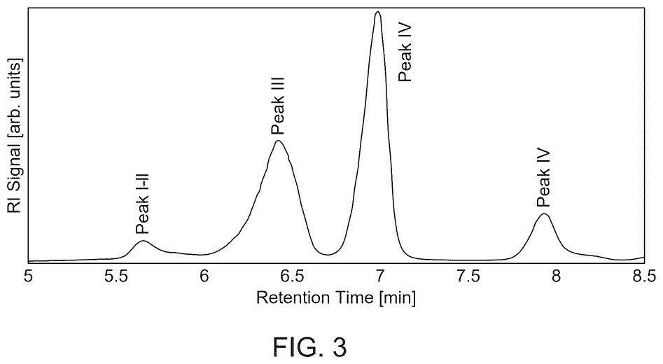

Chromatograms are processed and peaks integrated, for example using suitable software such as Thermo Fisher Scientific Chromeleon Data System. The focus of this analysis is a set of four or five peaks appearing early in the chromatogram that correspond to five characteristic polymer populations. (Sharper peaks, generally with longer retention time, are understood to correspond to small molecules present in the extracted active and are disregarded in this analysis.) The peaks observed in the chromatogram are designated in order of appearance on the chromatogram as Peaks I-II (generally appears as a single peak) and Peaks III, IV and V. A representative chromatogram containing the abovementioned peaks is shown in FIG. 3. The area of Peaks I-II, III, IV and V correspond to the relative concentration of aluminum polymer populations present in the analyzed active in the injected sample. (It is noted that these five polymer populations are generally recognized as present. Swaile U.S. Pat. No. 6,149,897 refers to the peaks in the chromatograms as Peaks I-II, Peak III, Peak IV, and Peak V. Similarly, Gosling U.S. Pat. No. 4,359,456 refers to Peak I-II as “Band 1,” Peak III as “Band 2, Peak IV as “Band 3”, and Peak V as “Band 4.”

Peaks I-II, III, IV, and V are each integrated, and their peak areas (arbitrary units) are recorded. The ratios between combinations of peaks of interest may be calculated and expressed as a dimensionless ratio. For example, the ratio of the area of Peak IV to the area of Peak III is calculated and reported as the “Peak IV/III ratio” to the nearest 0.01.

### pH Test Method

The pH of particulate active premixes and/or the actives are determined by creating a 15% solution of the powder in deionized water and measuring with a calibrated glass electrode and meter such as a Mettler Toledo® SevenCompact pH S210 or equivalent.

For an antiperspirant product the pH can be determined as follows. First, if the product is an aerosol, the pH of product concentrate was determined by piercing a small hole in the product to allow the propellant to evaporate for at least 24 hours, then removing the valve assembly to allow removal of the product concentrate. 10 grams of the product or the product concentrate was then mixed with 20 grams of deionized water. After mixing vigorously to dissolve the active, the mixture was centrifuged at 10,000 rpm for 10 minutes to separate the aqueous layer from the insoluble portions of the product concentrate. The pH of the aqueous phase is then determined using the same equipment as the active or active premix samples and reported as the pH of the product or product concentrate.

### Water Vapor Sorption Test Method

The Water Vapor Sorption Test Method is used to quantify the amount of water vapor uptake by a sample or composition when contained in a warm, moist environment. This method makes use of a dynamic vapor sorption (DVS) instrument to first dry and condition a specimen of a sample active premix or composition and then measure the increase of mass over time as the sample is held at elevated temperature and relative humidity.

A DVS instrument suitable for this method is capable of controlling percent relative humidity (% RH) to within ±3% RH, temperature to within ±2° C., and measuring mass to a precision of ±0.01 mg. It is further capable of maintaining environments of 0% RH at both 37° C. and 50° C. and 75% RH at 37° C. One suitable exemplary apparatus is the SPSx Vapor Sorption Analyzer (ProUmid GmbH & Co. KG, Ulm, Germany), or equivalent. The DVS instrument is configured to record the mass of specimen at ten-minute intervals with a precision of 0.01 mg or better.

A 30.0±5.0 mg specimen of material is spread evenly on a tared aluminum sample pan appropriate for the DVS instrument. The specimen and pan are introduced into the DVS, and the specimen is first dried at 50° C. and 0% RH for 10 hours. The specimen is then conditioned at 37° C. and 0% RH for 20 hours. The specimen mass point recorded at 37° C. and 0% RH is taken as the initial specimen mass, denoted mᵢ. The specimen is then exposed to an environmental state at 37° C. and 75% RH for 18 hours. For any point in time t during the final 18 hours of the measurement, the percent change in mass of the specimen is calculated by the expression:

$$Percent change in mass [ \% ] = \frac{{m}_{t} - {m}_{i}}{{m}_{i}} \times 100 \%$$

where mₜ is the measured specimen mass at that point in time, and time t is the total time passed after the beginning of the first, drying condition of the DVS. The maximum percent change in mass of the specimen observed over the final 18 hours of the measurement, corresponding to time 30 hours to 48 hours of the DVS measurement procedure overall, is reported as the Change in Mass of the specimen, reported in percent to the nearest 1%.

### Combinations:

A. A particulate active premix composition comprising:
   a. basic aluminum chloride of formula Al₂(OH)_{5.05}Cl_{0.95} to Al₂(OH)_{4.95}Cl_{1.05};
   b. calcium chloride;
   c. glycine;
   d. greater than 1% of an alkali metal salt and/or magnesium chloride;
      wherein the composition is a particulate.
B. The composition according to Paragraph A, wherein the calcium to aluminum atomic ratio is greater than 0.05, preferably greater than or equal to 0.07, more preferably greater than or equal to 0.09, and even more preferably greater than or equal to 0.10.
C. The composition according to Paragraphs A-B, wherein the calcium to aluminum atomic ratio is less than or equal to 1, preferably less than or equal to 0.75, preferably less than or equal to 0.5, preferably less than or equal to 0.3, and preferably less than or equal to 0.2.
D. The composition according to Paragraph A, wherein the calcium to aluminum atomic ratio is about 0.05 to about 0.3, preferably about 0.07 to about 0.25, more preferably from about 0.08 to about 0.23, and even more preferably from about 0.1 to about 0.2.
E. The composition according to Paragraphs A-D, wherein the composition comprises up to 25% of the alkali metal salt, preferably up to 20% alkali metal salt, more preferably up to 15% alkali metal salt, and most preferably up to 10% alkali metal salt.
F. The composition according to Paragraphs A-E, wherein the composition comprises an alkali metal salt chosen from sodium chloride, potassium chloride, lithium chloride, or mixtures thereof.
G. The composition according to Paragraph F, wherein the alkali metal salt comprises sodium chloride.
H. The composition according to Paragraphs A-G, having a glycine to calcium molar ratio is less than 2, preferably less than or equal to 1.75, preferably less than or equal to 1.5, more preferably less than or equal to 1.4, even more preferably less than or equal to 1.35, and most preferably less than or equal to 1.3.
I. The composition according to Paragraphs A-G, having a glycine to calcium molar ratio of greater than or equal to 0.25, preferably greater than or equal to 0.5, more preferably greater than or equal to 0.75, even more preferably greater than or equal to 0.85, more preferably greater than or equal to 0.9, even more preferably greater than or equal to 1.0, even more preferably greater than or equal to 1.2, and even more preferably greater than or equal to 1.27.
J. The composition according to Paragraphs A-G, having a glycine to calcium molar ratio of from about 0.5 to about 2, preferably from about 0.7 to about 1.8, more preferably from about 0.75 to about 1.75, even more preferably from about 0.8 to about 1.7, even more preferably from about 0.85 to about 1.6, even more preferably from about 0.9 to about 1.5, even more preferably from about from about 1 to about 1.45, even more preferably from about 1.2 to about 1.4, and most preferably from about 1.25 to about 1.35.
K. The composition according to Paragraphs A-G, having a glycine to calcium molar ratio of about 1.3.
L. The composition according to Paragraphs A-K, wherein the composition comprises a pH of about 4 to about 5.3, preferably from about 4.1 to about 5.2, more preferably about 4.2 to about 5.1, even more preferably from about 4.36 to about 5.0, and most preferably from about 4.4 to about 4.8, according to the pH Test Method.
M. The composition according to Paragraphs A-L, wherein the composition has a maximum change in mass of greater than or equal to 40% after 48 hours, preferably greater than or equal to 42%, preferably greater than or equal to 45%, more preferably greater than or equal to 50%, even more preferably greater than or equal to 52%, and even more preferably greater than or equal to 54%, according to the Water Vapor Sorption Test Method.
N. The composition according to Paragraphs A-M, wherein each particle comprises aluminum, chlorine, and sodium.
O. The composition according to Paragraph N, wherein the particle is homogenous.
P. The composition according to Paragraph N, wherein each particle further comprises a surface coating and at least a portion of the sodium is present in the surface coating, preferably >50% of the sodium is present in the surface coating, more preferably >75% of the sodium is present in the surface coating.
Q. The composition according to Paragraphs N-P, wherein at least a portion of the sodium is interspersed with the aluminum and chloride.
R. The composition according to Paragraphs A-Q, wherein the composition has a peak IV/III ratio of exceeding 0.75, preferably greater than or equal to 1, preferably greater than or equal to 1.5, preferably greater than or equal to 2, preferably greater than or equal to 2.5, preferably greater than or equal to 3, preferably greater than or equal to 3.25, preferably greater than or equal to 3.5, preferably greater than or equal to 3.75, and preferably greater than or equal to 3.85, according to the GPC Test Method.
S. The composition according to Paragraphs A-R, wherein the composition has a peak IV/III ratio of from about 0.75 to about 7.9, preferably from about 1.5 to about 5, more preferably from about 1.75 to about 4.5, more preferably from about 2 to about 4.25, more preferably from about 2.1 to about 4.1, even more preferably from about 2.25 to about 4, and even more preferably from about 2.4 to about 4.
T. The composition according to Paragraphs A-S, wherein the composition has a peak IV/III ratio of from about 2 to about 4, preferably from about 2.1 to about 3.75, more preferably from about 2.2 to about 3.5.
U. An antiperspirant product, wherein the product comprises the composition according to Paragraphs A-T, wherein the antiperspirant product is in the form of a spray, roll-on, cream, paste, foam, wipes, or stick.
V. The antiperspirant product according to Paragraph U, wherein the product further comprises a propellant and is in the form of a spray.
W. A method of making a particulate antiperspirant active premix according to Paragraphs A-T comprising:
   a. providing an aqueous solution of aluminum chlorohydrate with the formula Al₂(OH)_{5.05}Cl_{0.95} to Al₂(OH)_{4.95}Cl_{1.05};
   b. adding calcium chloride to provide a calcium to aluminum atomic ratio of at least 0.05 and adding glycine to provide a glycine to calcium molar ratio from about 1 to 2 to form an aqueous solution;
   d. adding sodium chloride at a concentration of more than 0.5% of the aqueous solution;
   e. after step (d), heating the aqueous solution at a temperature of more than 60° C. and less than 150° C., preferably less than 130° C., and more preferably less than 115° C. for more than 1 hour;
   f. after step (e), drying the aqueous solution to a plurality of particles to form a particulate antiperspirant active premix; and
   g. optionally grinding the particulate antiperspirant active premix to a Dv50 particle size less than 50 microns;
   h. optionally adding water to regenerate a solution comprising the aqueous premix solution.
X. A method of making a particulate antiperspirant active premix according to Paragraphs A-T comprising:
   a. providing an aqueous solution of aluminum chlorohydrate Al₂(OH)_{5.05}Cl_{0.95} to Al₂(OH)_{4.95}Cl_{1.05};
   b. adding calcium chloride to provide a calcium to aluminum atomic ratio of at least 0.05 and adding glycine to provide a glycine to calcium molar ratio from about 1 to 2 to form an aqueous solution;
   d. heating the aqueous solution at a temperature of more than 60° C. and less than 150° C., preferably less than 130° C., and more preferably less than 115° C. for more than 1 hour;
   e. after step (d), adding sodium chloride at a concentration of more than 0.5% of the aqueous solution;
   f. after step (e), drying, preferably spray drying, the aqueous solution to form a plurality of particles to form the particulate antiperspirant active premix; and
   g. optionally grinding the particulate antiperspirant active premix to a Dv50 particle size less than 50 microns;
   h. optionally adding water to regenerate a solution comprising the aqueous premix solution.
Y. A non-therapeutic method of reducing perspiration from the surface of the human body, comprising a step of topical application of a product comprising the particulate active premix composition according to Paragraphs A-S.
Z. The method of Paragraph Y, wherein said composition is applied to the underarms.
AA. A non-therapeutic use of an antiperspirant product comprising the particulate active premix composition according to Paragraphs A to S for reduction of bodily perspiration.
BB. Non-therapeutic cosmetic use of the particulate active premix composition according to Paragraphs A to S as an antiperspirant agent.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A particulate active premix composition comprising:
a. basic aluminum chloride of formula Al₂(OH)_{5.05}Cl_{0.95} to Al₂(OH)_{4.95}Cl_{1.05};
b. calcium chloride;
c. glycine;
d. greater than 1% of an alkali metal salt and/or magnesium chloride;
wherein the composition comprises a plurality of particles.

2. The composition of claim 1, wherein the calcium to aluminum atomic ratio is greater than 0.05.

3. The composition of claim 1, wherein the composition comprises up to 25% of the alkali metal salt.

4. The composition of claim 3, wherein the composition comprises an alkali metal salt chosen from sodium chloride, potassium chloride, lithium chloride, or mixtures thereof.

5. The composition of claim 4, wherein the alkali metal salt comprises sodium chloride.

6. The composition of claim 1, wherein the glycine to calcium molar ratio is less than 2.

7. The composition of claim 6, wherein the glycine to calcium molar ratio is less than or equal to 1.75.

8. The composition of claim 7, wherein the glycine to calcium molar ratio is less than or equal to 1.5.

9. The composition of claim 1, wherein the composition comprises a pH of about 4.0 to about 5.25.

10. The composition of claim 9, wherein the composition comprises a pH of about 4.25 to about 5.0.

11. The composition of claim 1, wherein the composition has a change in mass greater than or equal to 40% over 48 hours according to the Water Vapor Sorption Test Method.

12. The composition of claim 1, wherein the composition has a peak IV/III ratio of exceeding 0.75.

13. The composition of claim 1, wherein each particle comprises aluminum, chlorine, and sodium.

14. The composition of claim 13, wherein each particle further comprises a surface coating and at least a portion of the sodium is present in the surface coating.

15. The composition of claim 13, wherein the sodium is interspersed with the aluminum and chloride.

16. An antiperspirant product, wherein the product comprises the composition of claim 1, wherein the antiperspirant product is in the form of a spray, roll-on, cream, paste, foam, wipes, or stick.

17. The antiperspirant product according to claim 16, wherein the product further comprises a propellant and is in the form of a spray.

18. A method of making a particulate antiperspirant active premix comprising:
a. providing an aqueous solution of aluminum chlorohydrate with the formula Al₂(OH)_{5.05}Cl_{0.95} to Al₂(OH)_{4.95}Cl_{1.05};
b. adding calcium chloride to provide a calcium to aluminum atomic ratio of at least 0.05 and adding glycine to provide a glycine to calcium molar ratio from about 1 to 2 to form an aqueous solution;
c. adding sodium chloride to the aqueous solution at a concentration of more than 0.5% to form the aqueous premix solution;
d. heating the aqueous premix solution at a temperature of more than 60° C. for more than 1 hour;
e. after step (d), drying the aqueous premix solution to a plurality of particles, forming the particulate antiperspirant active premix; and
f. optionally grinding the particulate antiperspirant active premix to a Dv50 particle size less than 50 microns;
g. optionally adding water to regenerate a solution comprising the aqueous premix solution.

19. A method of making a particulate antiperspirant active premix comprising:
a. providing an aqueous solution of aluminum chlorohydrate Al₂(OH)_{5.05}Cl_{0.95} to Al₂(OH)_{4.95}Cl_{1.05};
b. adding calcium chloride to provide a calcium to aluminum atomic ratio of at least 0.05 and adding glycine to provide a glycine to calcium atomic ratio from about 1 to 2 to form an aqueous solution;
c. heating the aqueous solution at a temperature of more than 60° C. for more than 1 hour;
d. after step (c), adding sodium chloride at a concentration of more than 0.5% of the aqueous solution;
e. drying the aqueous solution to a particle, forming the particulate antiperspirant active premix; and
f. optionally grinding the particulate antiperspirant active premix to a Dv50 particle size less than 50 microns;
g. optionally adding water to regenerate a solution comprising the aqueous premix solution.
